(19) United States
(12) Patent Application Publication
(10) Pub. No.: US 2015/0359739 A1
(43) Pub. Date: Dec. 17, 2015

(54) EDIBLE FILM-STRIPS FOR IMMEDIATE RELEASE OF ACTIVE INGREDIENTS

(71) Applicant: Johnson & Johnson Consumer Inc., Skillman, NJ (US)

(72) Inventors: Frank J. Bunick, Randolph, NJ (US); David W. Wynn, Huntingdon Valley, PA (US); Der-Yang Lee, Flemington, NJ (US)

(21) Appl. No.: 14/836,412

(22) Filed: Aug. 26, 2015

Related U.S. Application Data
(62) Division of application No. 12/361,144, filed on Jan. 28, 2009.

(51) Int. Cl. A61K 9/00; A61K 9/70

(52) U.S. Cl. CPC A61K 9/0056; A61K 9/7007

(57) ABSTRACT

The present invention is directed to an edible film that contains two or more segmented portions and comprises an active ingredient that is distributed on a segmented portion or portions which comprise less than 50% of the cross sectional surface area of a major face of said film. The present invention also relates to methods of treatment for the treatment of various conditions and for taste-masking of pharmaceutical ingredients.

The present invention is directed to edible film-strips containing active ingredients with the ability to deliver such active ingredients in preferential configurations and methods. The configurations and methods disclosed herein demonstrate that the film may have an improved taste, deliver a modified release of the active ingredient, or target delivery of the active ingredient to affected areas.

### BACKGROUND

It is known to administer pharmaceutical active ingredients using solid, edible film-strips.

U.S. Pat. No. 7,025,983 discloses films, including edible films. The films include a water-soluble film-forming polymer such as pullulan. Edible films are disclosed that include pullulan and anti-microbially effective amounts of the essential oils thymol, methyl salicylate, eucalyptol and menthol. The edible films are said to be effective at killing the plaque-producing germs that cause dental plaque, gingivitis and bad breath. The film can also contain pharmaceutically active agents. Methods for producing the films are also disclosed.

Published PCT Application WO 2004/039166 discloses disintegrating or dissolving edible strips for use as a matrix for retaining and delivering nutrients, flavors and medicinal compounds that are made from liquid film casting compositions comprising a major proportion of gelatin. The particularly low melting range for hydrated gelatin produces films are said to leave virtually no residue upon dissolving in the mouth and can be used in the form of thicker films and strips than known edible films.

U.S. Pat. No. 7,067,166 discloses physiologically acceptable films, including edible films. The films include a water-soluble film-forming polymer, such as pullulan, and a taste masked pharmaceutically active agent, such as dextromethorphan. The taste-masking agent is preferably a sulfonated polymer ion exchange resin comprising polystyrene cross-linked with divinylbenzene, such as Amberlite. Methods for producing the films are also disclosed.

Published PCT Application WO 2004/096193 describes a consumable film that is adapted to adhere to and dissolve in the oral cavity of a warm-blooded animal including humans. The film comprises a modified starch, pharmaceutically active agent and, optionally, at least one water-soluble polymer.

Published PCT Application WO 2004/012720 describes a process for making rapidly dissolving and dispersing dosage forms, particularly orally consumable films, for the delivery of pharmaceutically active agents and with the dosage forms so obtained. The process comprises the steps of (a) preparing a hydrated polymer composition comprising pullulan and sodium alginate having a viscosity suitable for casting; (b) casting said composition into the shape of a dosage form; and (c) drying said dosage form under such conditions as to provide a form which rapidly dissolves and disperses in the mouth of the consumer.

Published PCT Application WO 2005/039499 describes disintegratable films containing a mixture of high molecular weight and low molecular weight water-soluble components; and a pharmaceutically or cosmetically active ingredient. The films optionally contain a starch component, a glucose component, a filler, a plasticizer and/or humectant. The films are preferably in the form of a mucoadhesive monolayer having a thickness sufficient to rapidly disintegrate in the oral environment and release the active ingredient without undue discomfort to the oral mucosa. The monolayer can be cut to any desired size or shape to provide conveniently useable unit dosage forms for administration to oral or other mucosal surfaces for human pharmaceutical, cosmetic, or veterinary applications. The invention further provides methods of administering the film compositions by placing the composition into, for example, the oral cavity for a sufficient period of time to permit the film to disintegrate and release the active ingredient.

Published U.S. Patent Application 2004/0247649 describes various edibles, their compositions, and manufacturing methods. Some examples of the edibles include orally soluble films. Some of the films may have a pleasant taste, carry nutraceuticals, carry medication, or serve other purposes.

Published U.S. Patent Application 2005/0163830 describes thin film-shaped or wafer-shaped pharmaceutical preparations for oral administration of active substances. The preparations contain at least one matrix-forming polymer which has at least one active substance and at least one carbon dioxide-forming substance dissolved or dispersed therein.

Published U.S. Patent Application 2004/0115137 describes films, such as water-soluble films. The films include a water-soluble film-forming polymer such as methyl hydroxypropylcellulose and/or sodium alginate. Edible films are disclosed that include methyl hydroxypropylcellulose and/or sodium alginate, emulsifier, breath freshening agents, stabilizing agents, plasticizers, surfactants, disintegrants, and preservatives. The edible films may be used to deliver an effective amount of an agent for killing bacteria that causes such maladies as dental plaque, gingivitis, bad breath, or the like. The film may optionally contain pharmaceutically active agents.

Published PCT Application WO 2006/047365 describes pharmaceutical compositions suitable for oral administration in the form of edible films comprising diclofenac.

Published PCT Application WO 2005/009386 describes rapidly dissolving, oral film preparations for rapid release of an active agent in the oral cavity, in particular, rapidly dissolving oral films comprising a nicotine active which achieve good transbuccal absorption and provide nicotine craving relief to an individual are disclosed herein.

Published PCT Application WO 2004/045537 describes an edible film comprising an active ingredient for relief of a cough or pharyngitis. The edible film comprising a film former and an active ingredient wherein the active ingredient can be selected from active ingredients having the desired effect of treating cough or pharnyngitis. Specific formulations for said film are also disclosed.

Published PCT Application WO 2004/0052853 describes pectin films that are treated to alter their dissolution characteristics. More specifically, the films can be made to dissolve more quickly by reducing the molecular weight of the starting pectin. Applications of the pectin films include drug delivery and breath films.

U.S. Pat. No. 6,824,829 describes a method of forming a thin film-strip. The method comprises coating a liner substrate with a wet slurry of film forming ingredients and drying the wet slurry in a drying oven to form a film. The moisture content of the film is measured as the film exits the drying oven and the film is rewound on itself. The rewound film is then stored in a minimal moisture loss environment during a curing process.

Published PCT Application WO 2005/115110 discloses an apparatus and method for forming a polymer film and/or oral dosage form having an active content, such as a vitamin, that is said to be a relatively high proportion of the total dry weight percent without being unpleasant to taste, leaving a bitter after taste, having poor mouth feel and/or being slow to dissolve.

Published U.S. Patent Application No. 2005/196354 relates generally to film compositions for use in the delivering topical and/or systemic actives, and more particularly to a slow dissolving or disintegrating strips, especially for delivering oral agents to the teeth and gums.

Published U.S. Patent Application No. 2006/073190 relates to a method of making a confectionery packet or sachet formed with an edible film and enclosing a center composition. The packet or sachet can be designed to be placed in the mouth, where the film dissolves and the center composition is released. In preferred embodiments, the center composition comprises a sugar alcohol, such as xylitol, that creates a cooling sensation. Many other flavors and/or colors or sensate can also be used in the center composition, and some embodiments include breath-freshening, anti-bacterial, nutraceuticals, or pharmaceutical compositions in the center composition. The invention also comprises the edible packets or sachets, especially those composed of film with a desired retained water level suitable for producing a self-sealing film and/or an edible film packet that is stable at room temperature for at least six to twelve months.

Published PCT Application WO 2006/119286 discloses a composition comprising a film layer wherein the film layer rapidly dissolves in an oral cavity and a coating comprising a powder matrix, wherein the coating is applied to at least one side of the film layer and wherein the powder matrix comprises a nutritional supplement, an adhesive, a bulking agent, a flow agent, and a sweetener.

Published U.S. Patent Application 2005/281757 discloses a composition for delivery of an oral care substance to a dental surface upon application of the composition thereto. The composition comprises a flexible film comprising the oral care substance dispersed in a film-forming effective amount of a polymeric matrix having a hydrophilic component, e. g., vinylpyrrolidone (VP), and a hydrophobic component, e.g., vinyl acetate (VA), in a weight ratio selected such that the film is substantially dissolvable in saliva in a period of time effective for delivery of the oral care substance. The polymeric matrix illustratively comprises a poly(VP/VA) copolymer having a VP/VA weight ratio of about 90:10 to about 10:90.

Published U.S. Patent Application 2004/258630 discloses an orally consumable film composition for delivering antiplaque and breath freshening benefits to the oral cavity which is rapidly dissolvable or dispersible in the oral cavity. The composition comprises a homogeneous mixture of a water-soluble or dispersible film forming polymer and a selected antibacterial ester.

Published PCT Application WO 2004/060298 discloses a dosage unit having a substrate comprising a first polymer; a deposit, including an active ingredient; and a cover layer comprising a second polymer, wherein the cover layer covers the deposit and is joined to the first surface of the substrate by a bond that encircles the deposit and wherein at least one of the first and second polymers is a graft co-polymer. The dosage unit wherein said first and second polymers may be the same, and also the graft co-polymer may be a polyvinyl alcohol-polyethylene glycol graft co-polymer. Also disclosed is a dosage unit wherein the deposit is formed on the substrate by electrostatic dry drug deposition. The dosage unit may also include a polymer that is a graft co-polymer; and an active ingredient, and the graft co-polymer may be polyvinyl alcohol-polyethylene glycol.

Published PCT Application WO 2004/009050 discloses an orally consumable film composition for delivering breath freshening agents to the oral cavity which is rapidly dissolvable or dispersible in the oral cavity. The composition comprises a homogeneous mixture of a water dispersible film forming polymer and an enzyme.

Published PCT Application WO 2003/101420 relates to a film-shaped preparation that is dissolvable in an aqueous media and is used to administer substances into the human or animal body. The preparation contains at least one water-soluble polymer. The invention is characterized in that the preparation contains one or several components that produce a gas under the effect of humidity or in the presence of an aqueous medium or when high temperature modifications occur.

U.S. Pat. No. 6,596,298 discloses films, including edible films. The films include a water-soluble film-forming polymer such as pullulan. Edible films are disclosed that include pullulan and antimicrobially effective amounts of the essential oils thymol, methyl salicylate, eucalyptol and menthol. The edible films are effective at killing the plaque-producing germs that cause dental plaque, gingivitis and bad breath. The film can also contain pharmaceutically active agents. Methods for producing the films are also disclosed.

Published PCT Application WO 2001/070194 discloses films, including edible films. The films include a water-soluble film-forming polymer, such as pullulan, and a taste masked pharmaceutically active agent, such as dextromethorphan. The taste-masking agent is preferably a sulfonated polymer ion exchange resin comprising polystyrene cross-linked with divinylbenzene, such as AMBERLITE. Methods for producing the films are also disclosed.

JP 2004/350024 relates to an orally administering preparation that is improved for ease for swallowing, easiness and safety on taking and a masking effect of the taste, smell, etc., of a medicine. The preparation has a medicine-containing layer, water-swelling gel-forming layers, a middle layer installed between the medicine-containing layer and water-swelling gel-forming layer, and a middle layer installed between the medicine-containing layer and the water-swelling gel-forming layer is provided with that the medicine-containing layer contains a hardly water-soluble polymer as a base agent, the middle layers contain a polyvinylpyrrolidone and the water swelling gel-forming layers are installed in a state that each of them are directly laminated with the middle layers at the outermost layer of the orally administering preparation.

Published PCT Application WO 2005/110358 relates to film-shaped medicaments for oral administration, in particular through the mouth, for treating climacteric disturbances are disclosed. The medicaments contain as active substance estriol and/or at least one pharmacologically acceptable estriol ester, alone or in combination with at least one gestagen.

JP 2005/232072 relates to a film preparation and a film food that is stable both under high and low humidity without impairing quick solubilities inherent in them. The film preparation and the film food are obtained by using methyl cellulose or hydroxypropyl methyl cellulose as film base substantially without any saccharides.

Published PCT Application WO 2003/070227 relates to a thin film-type or wafer-type medicinal preparation for the oral administration of active ingredients. Said preparation is characterized in that it contains at least one matrix-forming polymer in which at least one active ingredient and at least one carbon-dioxide-forming agent are dissolved or dispersed.

### SUMMARY OF THE INVENTION

The present invention is directed to an edible film-strip comprising two or more segmented portions, wherein a therapeutic active ingredient is distributed on at least one of the segmented portions. In one embodiment, the active is present in less than 50% of the total cross sectional surface area of a major face of said film. The segmented portion can comprise, within a part of said segmented portion of a length of at least 2 millimeters to a maximum of at least 6 millimeters, a concentration of active ingredient that is 10 percent greater by weight of total active than in a separate part equal in length from a separate portion of the film. In an alternative embodiment, one or more segmented portions of the film are substantially free of active ingredient.

The present invention is also directed to an edible film-strip comprising first and second portions and first and second active ingredients wherein the first active ingredient has a higher bitterness level than the second active ingredient and is contained only on the first portion of the edible film-strip and the second active ingredient is contained only on the second portion of the edible film-strip. The film can be tapered such that the second portion has a width on its major face that is less than 90% of the width of the first portion of its major face.

The present invention is also directed to a method of delivering a topical analgesic or topical anesthetic to the throat using an edible film by ingesting an edible film-strip comprising two or more segmented portions, a topical analgesic or topical anesthetic that is distributed on one of the segmented portions, wherein less than 50% of the cross sectional surface area of a major face of said film comprises the topical analgesic or topical anesthetic.

The present invention is also directed to a method of delivering a topical analgesic or topical anesthetic to the throat using an edible film by ingesting an edible film-strip comprising two or more segmented portions, a topical analgesic or topical anesthetic that is distributed on one of the segmented portions, wherein one segmented portion comprises, within a part of said segmented portion, of a length of at least 2 millimeters to a maximum of about 6 millimeters, a concentration of topical analgesic or topical anesthetic that is 10 percent greater by weight of total active than a separate part equal in length from a separate portion of the film. The portion of the edible film not containing a topical anesthetic can include a second therapeutic active ingredient.

The topical anesthetic can be selected from the group consisting of menthol, dyclonine, phenol, benzocaine, benzyl alcohol, hexylresorcinol and combinations thereof. More preferably, the topical analgesic is selected from the group consisting of ibuprofen, ketoprofen, acetaminophen, naproxen, diclofenac and combinations thereof. The topical anesthetic can be used to treat pharnyngitis.

The present invention is also directed to a method of delivering a topical soothing agent to the throat using an edible film using an edible film by ingesting an edible film-strip comprising two or more segmented portions, a topical soothing agent that is distributed on one of the segmented portions, wherein less than 50% of the cross sectional surface area of a major face of said film comprises the topical soothing agent. The topical soothing agent can be pectin.

The present invention is also directed to an edible film-strip having a first portion and a second portion and at least different two pharmaceutically active ingredients, wherein the first pharmaceutically active ingredient is contained on the first portion that provides an immediate release portion of the first pharmaceutically active ingredient in a dissolution medium and the second active ingredient is contained on the second portion that provides a modified release of the second pharmaceutically active ingredient in a dissolution medium, wherein the second portion detaches from the first portion upon ingestion. The film can be tapered such that the second portion has a width on its major face that is less than 90% of the width of the first portion of its major face. The second layer can include a therapeutic active ingredient. The film can include microgel liquid filled beads, wherein the liquid filled beads can be substantially free of therapeutic active ingredient or a second active ingredient

The present invention is also directed to an edible bilayer film-strip, wherein the second layer comprises a therapeutic active ingredient. The first layer can be substantially free of a therapeutic active ingredient. The first layer and second layer can have the same active ingredient, but the second layer comprises a different amount of active ingredient than the amount in the first layer.

The present also relates to a method of reducing the bitterness of at least one active ingredient by using the edible films described herein.

### BRIEF DESCRIPTION OF DRAWINGS

FIG. 1 is a top view of an edible film-strip having two distinct portions or segments.

FIG. 2 is a top view of an edible film-strip in which an active ingredient is portioned in a gradient pattern over the cross-sectional area of the strip.

FIG. 3 is a top view of an edible film-strip in which one or more active ingredients are positioned on its major face.

FIG. 4 is a side view of an edible film-strip in which actives are separated into two different portions relative to a vertical axis.

FIG. 5 is a side view of an edible film-strip with an upper portion and a lower portion.

FIG. 6 is a top view of an edible film-strip having a selected shape.

FIG. 7 is a top view of an edible film-strip having a detachable portion.

FIG. 8 is a top view of an edible film-strip having embedded beads.

FIG. 9 is a top view of an edible film-strip having distinct portions for a single active ingredient.

FIG. 10 is a top view of an edible film-strip having a tapered shape.

### DETAILED DESCRIPTION OF THE INVENTION

The present invention is directed to various forms of improved edible strips for the delivery of one or more pharmaceutically active ingredients. One embodiment of the invention is directed to an edible dosage form that contains active ingredient, which is incorporated only on one side of an edible film-strip. This placement of active ingredient allows the active ingredient to be placed into the mouth with a section that avoids contact with much of the surface of the tongue, creating a superior tasting product.

The active ingredient can be placed in a separate solution stream during manufacturing and combined during rolling, or strategically sprinkled into one portion of the strip before drying and cutting. The active could also be added in the form of resin based particles or coated particles.

Active ingredients can have different types of adverse tastes, including bitterness, sourness, burning as often associated with propionic acids such as ibuprofen or ketoprofen, and or chalkiness as often associated with antacids such as calcium carbonate or aluminum hydroxide. Active ingredients can also impart adverse texture experiences when ingested depending on particle size or shape. In addition, certain types of particle coating materials such as insoluble coatings comprising ethylcellulose, methacrylates or cellulose acetates (cellulose acetate, cellulose acetate butyrate) can impart a gritty texture.

As used herein, “immediate release” means that the dissolution characteristics of at least one active ingredient meets USP specifications for immediate release tablets containing that active ingredient. An active ingredient having an immediate release property may be dissolved in the gastrointestinal contents, with no intention of delaying or prolonging the dissolution of the active ingredient. For example, for acetaminophen tablets, USP 24 specifies that in pH 5.8 phosphate buffer, using USP apparatus 2 (paddles) at 50 rpm, at least 80% of the acetaminophen contained in the dosage form is released therefrom within 30 minutes after dosing, and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes after dosing. See USP 24, 2000 Version, 19-20 and 856 (1999). Additionally, ibuprofen suspension may be analyzed for dissolution using pH 5.6 acetate buffer using USP apparatus 2 (paddles) at 50 rpm, where at least 80% of the ibuprofen contained in the dosage form is released therefrom within 60 minutes after dosing for an immediate release dose.

As used herein a “therapeutic active ingredient” is one which delivers a therapeutic benefit such as a pharmaceutical active ingredient, a vitamin supplement or a nutraceutical, not including active agents such as flavoring agents, sweeteners, or salivation inducing agents.

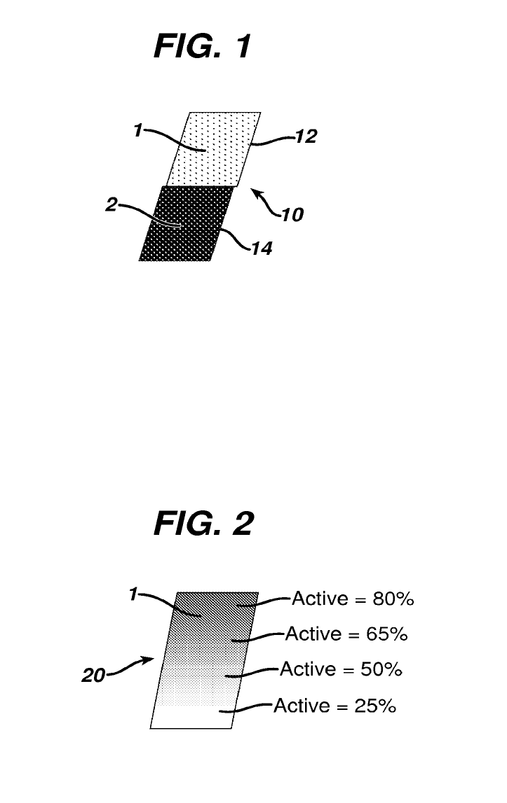

FIG. 1 illustrates an edible strip 10 having distinct first portion 12 and second portion 14 wherein a first active ingredient 1 is separated from a second active ingredient 2 by providing such active ingredients only on first portion 12 and second portion 14, respectively. First portion 12 and second portion 14 are separated from one another by a perforated line or some other means, such as color, to visually highlight the separate nature of these portions. In one version of this embodiment, the active ingredient(s) are separately added to two portions of a wet film from an external dosing mechanism, such as a powder feeder. In another version of this embodiment, the one active ingredient is added to one film composition as a solution or suspension, and the second active ingredient is added to a second film composition as a solution or suspension; and the two edible film compositions are combined and dried together. In one embodiment, when the two edible film compositions are dried together, an overlap of the two film portions exists which is from about 1 millimeter to about 15 millimeters mm in width, or about 1 millimeter to about 5 millimeters in width.

In alternative embodiment, one active ingredient is placed on the front part of the edible film and a second, more bitter active or active with a poorer taste profile is placed on the back part of the strip and is ingested and swallowed more rapidly. Advantageously, this type of strip could also be used as a means for separating two or more incompatible active ingredients. The terms “front” and “back” refer to relative positioning in the consumer's mouth. Bitterness can be quantified and compared using an Alpha MOS Electronic tongue using a bitterness intensity prediction model as compared to a placebo.

In one embodiment, the edible film-strip comprises one or more segmented portions that contain the active ingredient. The segmented portions which contain active ingredients can comprise 50 percent or less of the cross sectional surface area of one of the major faces of the film. The cross sectional surface area of the film face or film portion face is defined by the calculation of the length×width of any one face portion, or of the entire film face, when the film face or film portions face is in the shape of a rectangle, square, or parallelogram. The length and width are defined as the two longest axes of a three dimensional object, and does not include the height of the object. When the cross sectional area of a film or film portion in the shape of a trapezoid, the cross-sectional surface area is equivalent to [(0.5×height)×(length of base side 1+length of base side 2)].

A “major face” is defined herein as the top or bottom of the film, wherein the cross-sectional area of the face is defined by the length×width of the film. A “minor face” is defined herein by side of the edible film, measured as the height of the film, wherein the cross-sectional area is defined by the length×height, or the width×height.

In one embodiment, a second segmented portion 14 is substantially free of active ingredients, defined herein as less than 2 percent by weight of the dried film portion. In one embodiment, the second segmented portion 14 comprises a second active ingredient.

In one embodiment shown in FIG. 2, first active ingredient 1 is apportioned in an increasing gradient pattern across the major face of edible strip 20 so that there is a greater concentration of first active ingredient 1 in one section of the edible strip 20 than in the remainder of edible strip 20. The sectional concentration variation of first active ingredient 1 allows edible strip 20 to be ingested, when positioned properly in the mouth, with less taste perception along the surface of the tongue. In one embodiment a second active ingredient may be present which is present equally throughout the surface area of the film. In one version of this embodiment, a flavoring agent 1 is apportioned in a gradient fashion along the film and the therapeutic active ingredient is equally distributed throughout the film.

In one embodiment illustrated in FIG. 3, a first active ingredient 1 is apportioned along the sides of edible strip 10 and a second active 2 is apportioned in the middle section of edible strip 30 to allow for separate dissolution of edible strip 30 along the surface of the tongue. In this embodiment greater than about 50 percent; e.g. greater than about 30 percent of the active ingredient is placed equally on the left, 25 percent or less of the film surface area, and on the right 25 percent or less of the film surface area. In embodiment wherein the more than one active ingredient is present in the film, the more bitter active ingredient is present on the side portions of the film. The less bitter active may be equally distributed throughout the film. In one version of this embodiment, a flavoring agent 1 is apportioned along the sides of the edible film, and the therapeutic active ingredient is equally distributed throughout the film.

In another embodiment (not shown), only one active is apportioned on either the side or middle section(s) of the strip. In another embodiment the level of active ingredient is apportioned in a gradient manner along the surface area of the film, wherein at least some portion of active is present within all areas of the film, but a greater portion is present on one side. The less bitter active may be equally distributed throughout the film.

In another embodiment, the active ingredient is present on the side portions of the film. In this embodiment greater than about 50 percent; e.g. greater than about 30 percent of the active ingredient is placed equally on the left 25 percent or less of the film surface area and on the right 25 percent or less of the film surface area. In embodiment wherein the more than one active ingredient is present in the film, the more bitter active ingredient is present on the side portions of the film. The less bitter active may be equally distributed throughout the film.

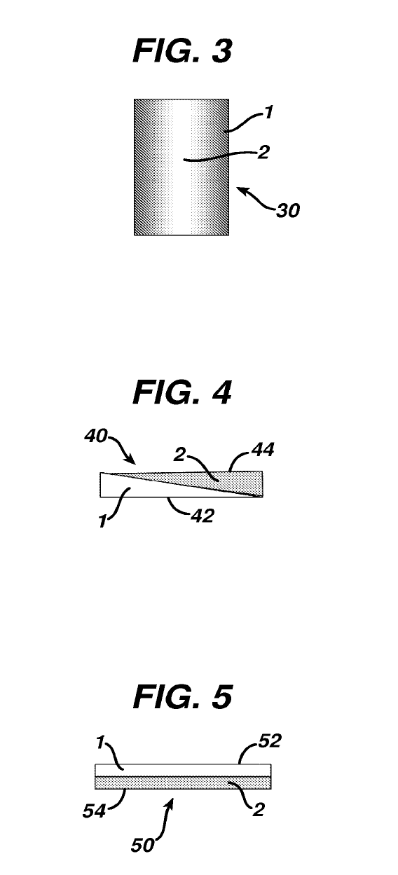

In an embodiment illustrated in FIG. 4, active ingredients are provided in first portion 42 and second portion 44 of edible strip 40 that are oriented on the vertical axis. Preferably, the portion including the active ingredient having a more bitter taste perception is placed away from the tongue such that dissolution of the bitter tasting active ingredient is delayed. In one embodiment second portion 44 is present as a modified release matrix comprising a second active ingredient.

In an embodiment illustrated in FIG. 5, an edible strip 50 is provided having an upper portion 52 and a lower portion 54. Active ingredients can be provided in either one or both portions of edible strip 50. In one version of this embodiment (not shown), lower portion 54 does not contain a therapeutic active ingredient, but shields the active on upper portion 52 from immediately contacting from the tongue. In one embodiment, the active ingredient is distributed such that a majority of the active is present on top third of the surface area of the film; for example, greater than about 50 percent; e.g. greater than about 30 percent of the active is present in the top third of the film. In embodiments wherein more than one active ingredient is present in the film, the more bitter active ingredient is present on the top portion of the film.

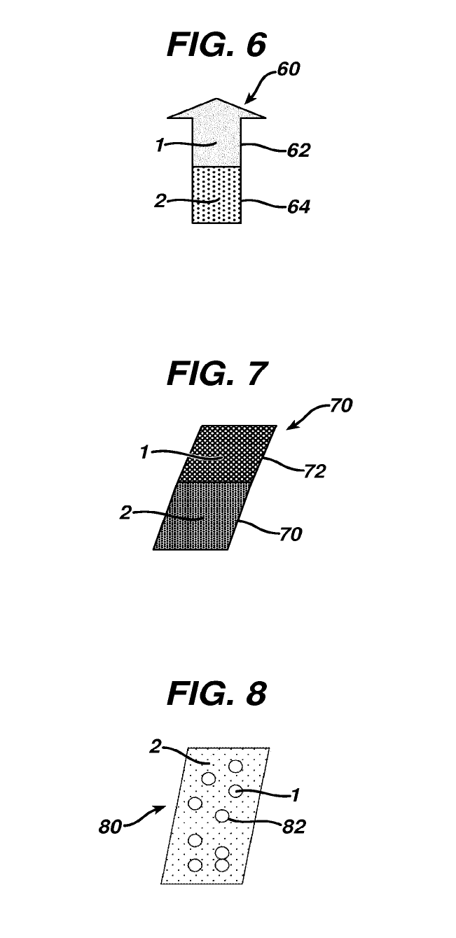

In one embodiment shown in FIG. 6, an edible strip 60 is provided having first portion 62 and second portion 64. First active ingredient 1 is separated from second active ingredient 2 on each of the respective portions, and the strip is intentionally shaped in a way (i.e. an arrow) so that the consumer or patient can be shown how to administer the strip so that the taste of the more bitter (or burning) tasting active is experienced only in the back area of the tongue. In one version of this embodiment, a topical anesthetic is placed on the back portion of the strip so that the topical anesthetic is administered more directly to affected throat areas. In one version of this embodiment a first flavoring agent 1 is present in first portion 62 and first portion 62 does not comprise a therapeutic active ingredient, and a therapeutic active ingredient 2 is present in second portion 64.

In another embodiment the edible film is shaped such that the user intuitively places the strip into mouth with the portion of the film containing a greater amount of the active ingredient. This can be achieved by tapering the film such that the larger surface area portion is placed into the mouth first. In another embodiment the film has an arrow head or round bud portion such that the large part if placed into the mouth in the indicated direction.

In one embodiment illustrated in FIG. 7, an edible strip 70 is provided with a detachable portion 72 and a second portion 74. First active ingredient 1 is placed onto detachable portion 72, which preferably releases first active ingredient 1 to produce a modified release profile in a dissolution medium, which would either be in the oral cavity or further within the gastrointestinal tract. The modified release portion may be in the form of a matrix, which dissolves in a modified release manner in the gastrointestinal tract. The modified release portion may also contain particles of active ingredient coated with a modified release coating. First active ingredient 1 can also be provided on second portion 72, which preferably releases first active ingredient 1 to produce an immediate release profile in a dissolution medium. In one version of this embodiment (not shown), the immediate release portion does not contain an active ingredient.

In an alternative embodiment, a small portion of the edible film-strip that contains a pharmaceutical active ingredient is a modified release type of matrix that is capable of separating from the main portion of the film-strip when ingested. This separated portion of the film-strip then slides down the back of the throat before releasing in a controlled release manner in the gastrointestinal tract. The separating portion could also incorporate a texture enhancer or wetting agent to facilitate swallowing.

As used herein, “modified release” shall apply to the altered release or dissolution of an active ingredient in a dissolution medium, such as gastrointestinal fluids. The active ingredient or ingredients that may be released in a modified manner may be contained within, for example, dosage forms, coatings, or particles, or in any portion thereof, such as, for example, particles dispersed throughout a liquid suspending medium. Types of modified release include: 1) extended release; or 2) delayed release. In general, modified release dosage forms are formulated to make the active ingredient(s) available over an extended period of time after ingestion, which thereby allows for a reduction in dosing frequency compared to the dosing of the same active ingredient(s) in a conventional dosage form. Modified release dosage forms also permit the use of active ingredient combinations wherein the duration of one active ingredient may differ from the duration of another active ingredient.

In the embodiment wherein the strip comprises a detachable modified release matrix portion, the matrix may comprise modified release water swellable polymers such as but not limited to high molecular weight grades of hypromellose including those commercially available from Dow Chemical as Methocel® K100, K4M, K15M, E4M, and E10M, hydroxypropyl cellulose including those commercially available from Hercules, Inc. as Klucel® LF, JF, and GF; cross linked gelatin, gums such as xanthan gum, locust bean gum, guar gum, mannan gum, gum arabic, gellan gum and thickeners such as carrageenan and pectin. In one embodiment the edible film comprises, by weight of the detachable modified release portion, from about 5 percent to about 50 percent; e.g. from about 10 percent to about 60 percent of one or more modified release water swellable polymers.

In one embodiment the modified release active ingredient may be coated with polymer systems that impart an enteric release for the active ingredient. In one embodiment, the modified release active ingredient may be present in a matrix that imparts an enteric release profile for the active ingredient. The matrix may be a detachable portion, as shown in FIG. 10, or as part of a bi-layer film. In one embodiment the matrix imparts an enteric release profile for the active ingredient. The matrix may also comprise enteric polymers such as, but are not limited to hydroxypropylmethylcellulose phthalate (also known as hypromellose phthalate), hydroxypropylmethylcellulose acetate succinate, cellulose acetate phthalate, polyvinylacetate phthalate, shellac, enteric polymethacrylate-based polymers, and copolymers and mixtures thereof.

Examples of suitable enteric polymethacrylate-based polymers include, but are not limited to poly(methacrylic acid, methyl methacrylate) 1:2, which is commercially available from Rohm Pharma GmbH under the tradename, “EUDRAGIT S” polymers; poly(methacrylic acid, methyl methacrylate) 1:1, which is commercially available from Rohm Pharma GmbH under the tradename, “EUDRAGIT L-100, L-30D, L 12.5 and L12.5 P” polymers; and poly(methacrylic acid, ethyl acrylate) 1:1 which is commercially available from Rohm Pharma under the tradename “EUDRAGIT L30-D 55 and L-100-55,” from Eastman Chemical under the tradename “Eastacryl 30D,” from Colorcon Corporation under the tradename, “Acryl-EZE” and from BASF Fine Chemicals under the tradename, “Kollicoat MAE 30D.”

In one embodiment, the enteric polymer may be selected from non-acrylate compounds, such as hydroxypropyl methylcellulose phthalate, hydroxypropyl methylcellulose acetate succinate, cellulose acetate phthalate, polyvinyl acetate phthalate, shellac and copolymers and mixtures thereof. In one embodiment the edible film comprises, by weight of the detachable modified release portion, from about 20 to about 80 percent; e.g. from about 20 percent to about 60 percent of one or more enteric polymers.

In one embodiment the modified release matrix portion comprises one active ingredient as part of the matrix and a second active ingredient that is coated with a modified release coating. In one embodiment the detachable modified release portion detaches since the immediate release portion of the edible film is more soluble in the mouth than modified release portion. In another embodiment the detachable modified release portion has a perforated lined between the immediate release portion and the modified release portion to facilitate detachment.

In one embodiment the immediate release edible film portion contains nicotine. In certain embodiments, the nicotine in any form is selected from the group consisting of a nicotine salt, the free base form of nicotine, a nicotine derivative, such as a 30 nicotine cation exchanger, a nicotine inclusion complex or nicotine in any non-covalent binding; nicotine bound to zeolites; nicotine bound to cellulose or starch microspheres; and mixtures thereof. Still, further the nicotine inclusion complex may be a cyclodextrin, such as p-cyclodextrin. Even further the cation exchanger may be a polyacrylate. Even more further, the nicotine salt may be a tartrate, hydrogen tartrate, citrate or maleate. The nicotine may act as a stimulant to obtain a rapid reduction of the urge to smoke or to use tobacco.

With nicotine it is intended to include nicotine, 3-(1-methyl-2-pyrrolidinyl) 10 pyridine, with its base form, including synthetic nicotine as well as nicotine extracts from tobacco plants, or parts thereof, such as the genus Nicotiana alone or in combination; or pharmaceutically acceptable salts.

In one embodiment the edible film incorporates nicotine as the free base form or as a water-soluble pharmaceutically acceptable salt, either per se or adsorbed on a adsorbent, or 15 as a complex with a cation exchanger or mixtures of the foregoing, as an inclusion complex, such as a cyclodextrin complex, e g p-cyclodextrin, but any other suitable pharmaceutically acceptable form may also be employed.

In one embodiment illustrated in FIG. 8, an edible strip 80 is shown having a plurality of embedded microgel liquid filled beads 82. The liquid filled beads 82 contain at least one active ingredient 1, while film-strip 80 optionally further comprises second active ingredient 2.

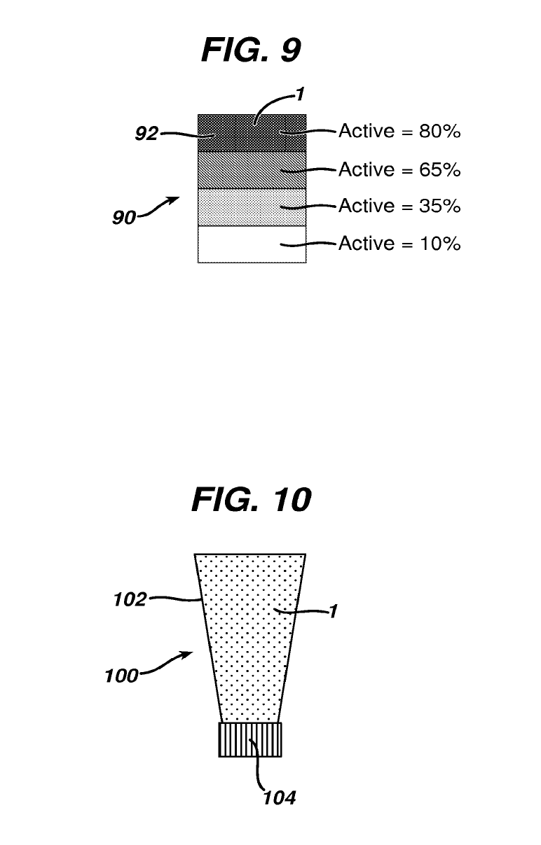

In one embodiment illustrated in FIG. 9, an edible film-strip 90 is provided with a first active ingredient 1 that is apportioned in a generally increasing step-wise gradient fashion with a plurality of segmented portions 92. Gradient portions 92 of first active ingredient 1 are separate and distinct from one another and are positioned along the length of edible strip 90 and one major surface thereof. In another version of this embodiment, a flavoring agent 1 is apportioned in a generally increasing step-wise gradient fashion and a therapeutic active ingredient is apportioned in a generally increasing step-wise throughout the film.

In one embodiment illustrated in FIG. 10, an edible strip 100 is provided having a tapered portion 102 that indicates the direction in which the edible film-strip is to be administered. In another embodiment, edible film-strip 100 has instead of or in addition to tapered portion 102, a textured portion 104 on one side for easier handling and gripping. In another embodiment, the edible film-strip has an edible handle that protrudes from the top of the package, wherein the user knows that this portion of the film-strip is to be held by the fingers, and subsequently, the opposite end is placed into the mouth and contains the higher level of active ingredient. This edible handle may be in any suitable shape, for example a ring or hollowed out portion, or as a ridged portion, facilitating the grip of the film.

The present invention also relates to a method for delivering a topical anesthetic to the back of the throat by providing the active on the back portion of the edible strip film and thereby targeting the delivery of the anesthetic to the throat only. Suitable actives for treating sore throat include topical anesthetics such as but not limited to dyclonine, benzocaine, and lidocaine; salts such as sodium chloride, potassium chloride and magnesium chloride; and soothing agents such as pectin. In this embodiment, the active is apportioned on the part of the strip that is in close proximity to the affected area of the throat.

The various embodiments described above are suitable for treating many upper respiratory conditions, including for example, acute viral pharyngitis. Treatment of this condition is usually symptomatic and consists mainly of rest, warm saline gargles, throat lozenges containing a mild anesthetic, at least 2 quarts of fluid daily, and analgesics as needed.

The invention provides a physiologically acceptable film that is particularly well adapted to adhere to and dissolve in a mouth of a consumer to deliver one or more pharmaceutically active ingredients to a consumer. Preferred films according to the invention comprise one or more pharmaceutically active agents that is (are) provided in selected locations on the film, a film-forming agent, and at least one of the following additional ingredients: water, antimicrobial agents, plasticizing agents, flavoring agents, saliva stimulating agents, cooling agents, surfactants, stabilizing agents, emulsifying agents, thickening agents, binding agents, coloring agents, sweeteners, fragrances, triglycerides, preservatives, polyethylene oxides, propylene glycol, and the like.

In one embodiment, the edible film delivers sequential flavors to the consumer, that is, the first flavor is perceptible to the consumer before the second flavor, or vice-versa. In one embodiment, for example, the consumer perceives the first flavor which is substantially absent of the second flavor for some period of time, then optionally the consumer perceives both flavors for a period of time, but at varying levels of intensity, then finally the consumer perceives the second flavor substantially absent of the first flavor for a period of time. In another embodiment, the consumer perceives both the first and second flavors initially, followed by a period of time during which the intensity of the first flavor decreases, and the patient continues to perceive the second flavor after the perception of the first flavor has diminished or ended. In one embodiment the first flavor may be present in one portion of the edible film and the second flavor may be present in a second portion of the edible film. In another embodiment, at least one flavor is distributed in a gradient fashion along the cross sectional surface area of the film, wherein the concentration is gradually increased or decreased across the length of the film. In one embodiment one flavor is present on one face of a bilayer edible film and a second flavor is present on the second layer of the edible film. On one embodiment one layer of a bilayer edible film comprises at least one active pharmaceutical agent and the second layer comprises a flavor, and is substantially free of the first active pharmaceutical agent.

For example, the flavoring agent may persist in the oral cavity until after all or substantially all of the edible film has been swallowed so that the patient continues to perceive the second flavor after the dosage form has been swallowed. The flavoring agent may be a solid of particular shape or other physical or chemical property that has a certain adhesion or surface tension in the oral cavity. In one particular embodiment, at least one flavoring agent is in the form of flaked films that become suspended in the edible film upon combination therewith. The flaked films, which preferably have a thickness of about 0.05 mm, coat the surfaces of the oral cavity and are held in place there until after all of the dosage form has been swallowed. The flaked films have a mean thickness of at least about 0.025 mm, e.g. at least about 0.04 mm. In one embodiment a first amount of flavoring agent is suspended or dissolved in the edible film as a particulate; and a second amount of flavoring agent is in the form of a flaked film, wherein the second amount may be the same or different flavor agent as in the first amount of flavoring agent.

Suitable flavoring agents are for example those proprietary blends of chemicals commercially available from various flavor companies, for example, International Flavors and Fragrances, Busch Boake Allen, and Firmenich. Typical flavors to be imparted by these flavoring agents include but are not limited to fruit flavors such as cherry, berry, citrus, apple, grape, watermelon, and the like; candy flavors such as chocolate, vanilla, caramel, bubblegum, cotton candy, and the like; and mint flavors such as peppermint, spearmint, cinnamon, menthol, and the like.

In another embodiment of the invention, the edible film also comprises a texturizing agent. Here, the edible film may initially have a smooth, gritty, or other first texture displayed from one portion of the film. A second portion of the film may comprise a separate texture because of a different concentration of the first texturizing agent or a different type of texturizing agent. The edible film may exhibit dual textures, that is, distinct regions of each texture, such as a swirl of two separate textures, or small or large areas of one texture within the other texture.

### Analysis of Active

The quantity of active ingredient in the edible film may be analyzed by a variety of means. In one embodiment, the quantity of active is calculated as area in a portion of the cross-sectional surface area. The particles which are present as a crystal, coated particle or bound to an ion exchange resin can be measured using light microscopy or scanning electron microscopy, wherein various portions of particles can be separated and measured for contribution to the total surface area.

In one embodiment, the segmented portions contain a concentration of active ingredient which is higher than another portion. In this embodiment the portion comprises, by weight within one part of one segmented portion of a length of at least about 2 millimeters to a maximum of 6 millimeters, a concentration which is 10 percent greater; e.g. 25 percent greater by weight of total active than a part of a separate portion of the film which is equal in length. Concentration is defined herein as the weight of active ingredient per unit weight of the edible film or film portion (i.e. mg active/mg edible film). In this embodiment, the active ingredient is measured by assay of the active in a cut-out portion of the film of said length, using typical assay techniques such as wet chemistry, microscopy, and liquid chromatography. In one embodiment the film and active ingredient are dissolved in a suitable media to perform the assay.

The expression “physiologically acceptable” as used herein is intended to encompass compounds, which upon administration to a patient, are adequately tolerated without causing undue negative side effects. The expression encompasses edible compounds.

The expression “pharmaceutically active agents” as used herein is intended to encompass agents other than foods, which promote a structural and/or functional change in and/or on bodies to which they have been administered. These agents are not particularly limited; however, they should be physiologically acceptable and compatible with the film. Suitable pharmaceutically active agents include, but are not limited to: antimicrobial agents, such as triclosan, cetyl pyridium chloride, domiphen bromide, quaternary ammonium salts, zinc compounds, sanguinarine, fluorides, alexidine, octonidine, EDTA, and the like;

non-steroidal anti-inflammatory drugs, such as aspirin, acetaminophen, ibuprofen, ketoprofen, diflunisal, fenoprofen calcium, naproxen, tolmetin sodium, indomethacin, and the like;
anti-tussives, such as benzonatate, caramiphen edisylate, menthol, dextromethorphan hydrobromide, chlophedianol hydrochloride, and the like;
decongestants, such as pseudoephedrine hydrochloride, phenylephrine, phenylpropanolamine, pseudoephedrine sulfate, and the like;
anti-histamines, such as brompheniramine maleate, chlorpheniramine maleate, carbinoxaminc maleate, clemastine fumaratc, dexchlorpheniramine maleate, diphenhydramine hydrochloride, diphenylpyraline hydrochloride, azatadine maleate, diphenhydramine citrate, doxylamine succinate, promethazine hydrochloride, pyril amine maleate, tripelennamine citrate, triprolidine hydrochloride, acrivastine, loratidine, brompheniramine, dexbrompheniramine, and the like;
expectorants, such as guaifenesin, ipecac, potassium iodide, terpin hydrate, and the like; anti-diarrheals, such a loperamide, and the like;
H2-antagonists, such as famotidine, ranitidine, and the like;
proton pump inhibitors, such as omeprazole, lansoprazole, and the like; general nonselective CNS depressants, such as aliphatic alcohols, barbiturates and the like; general nonselective CNS stimulants such as caffeine, nicotine, strychnine, picrotoxin, pentylenetetrazol and the like;
drugs that selectively modify CNS function, such as phenyhydantoin, phenobarbital, primidone, carbamazepine, ethosuximide, methsuximide, phensuximide, trimethadione, diazepam, benzodiazepines, phenacemide, pheneturide, acetazolamide, sulthiame, bromide, and the like;
Anti-parkinsonism drugs such as levodopa, amantadine and the like;
narcotic-analgesics such as morphine, heroin, hydromorphone, metopon, oxymorphone, levorphanol, codeine, hydrocodone, xycodone, nalorphine, naloxone, naltrexone and the like;
analgesic-antipyretics such as salycilates, phenylbutazone, indomethacin, phenacetin and the like; and psychopharmacological drugs such as chlorpromazine, methotrimeprazine, haloperidol, clozapine, reserpine, imipramine, tranylcypromine, phenelzine, lithium and the like.

In one particular embodiment, at least one active ingredient is selected from propionic acid derivative NSAID, which are pharmaceutically acceptable analgesics/non-steroidal anti-inflammatory drugs having a free —CH(CH₃)COOH or —CH₂CH₂COOH or a pharmaceutically acceptable salt group, such as —CH(CH₃)COO—Na+ or CH₂CH₂COO—Na+, which are typically attached directly or via a carbonyl functionality to a ring system, preferably an aromatic ring system.

Examples of useful propionic acid derivatives include ibuprofen, naproxen, benoxaprofen, naproxen sodium, fenbufen, flurbiprofen, fenoprofen, fenoprofen calcium, flurbiprofen, tiaprofenic, oxaprozin, fenbuprofen, ketoprofen, indoprofen, pirprofen, carpofen, oxaprofen, pranoprofen, microprofen, tioxaprofen, suprofen, alminoprofen, tiaprofenic acid, fluprofen, bucloxic acid, and pharmaceutically acceptable salts, derivatives, and combinations thereof.

In one embodiment of the invention, at least one active ingredient may be selected from bisacodyl, albuterol, famotadine, ranitidine, cimetidine, prucalopride, diphenoxylate, loperamide, lactase, mesalamine, bismuth, antacids, and pharmaceutically acceptable salts, esters, isomers, and mixtures thereof.

In another particular embodiment of the invention, at least one active ingredient may be selected from pseudoephedrine, phenylephrine, phenylpropanolamine, chlorpheniramine, dextromethorphan, diphenhydramine, clofedianol, astemizole, terfenadine, fexofenadine, loratadine, desloratadine, cetirizine, mixtures thereof and pharmaceutically acceptable salts, esters, isomers, and mixtures thereof.

In a particular embodiment the active ingredient in the modified release portion is selected from phenylephrine, pseudoephedrine, dextromethorphan, diphenhydramine, chlorpheniramine and mixtures thereof.

The amount of pharmaceutically active agent that can be used in the rapidly dissolving films, according to the present invention, is dependent upon the dose needed to provide an effective amount of the pharmaceutically active agent. Examples of doses for specific pharmaceutically active agents that can be delivered per one strip of rapidly dissolving oral film are reviewed in Table A.

TABLE A

| Active Ingredient | Preferred Dose | |
|---|---|---|
| Chlorpheniramine Maleate | 4 | mg |
| Brompheniramine Maleate | 4 | mg |
| Dexchlorpheniramine | 2 | mg. |
| Dexbrompheniramine | 2 | mg |
| Triprolidine Hydrochloride | 2.5 | mg |
| Acrivastine | 8 | mg |
| Azatadine Maleate | 1 | mg |
| Loratidine | 10 | mg. |
| Phenylephrine Hydrochloride | 10 | mg |
| Dextromethorphan Hydrobromide | 10 to 30 mg | |
| Ketoprofen | 12.5 to 25 mg | |
| Sumatriptan Succinate | 35 to 70 mg | |
| Zolmitriptan | 2.5 | mg |
| Loperamide | 2 | mg |
| Famotidine | 10 mg to 20 mg | |
| Nicotine | 2 | mg. |
| Diphenhydramine Hydrochloride | 12.5 to 25 mg | |
| Pseudoephedrine Hydrochloride | 30 | mg |

The active ingredients may be present in a crystalline or amorphous state. In one embodiment, first active ingredient is solubilized within the film materials, and second active ingredient is suspended. For suspended active ingredients, the mean particle size may be from about 1 micron to about 200 microns, e.g. from about 5 microns to about 70 microns.

In one embodiment, an antacid is present in the edible film-strip to treat esophageal reflux. Esophageal reflux can cause discomfort in the back of the throat, caused by acid that has traveled up through the throat. If the antacid is present at one end of a tapered film it may be used for targeted treatment of reflux. Suitable antacids include but are not limited to calcium carbonate, magnesium hydroxide, magnesium oxide, magnesium carbonate, aluminum hydroxide, sodium bicarbonate, dihydroxyaluminum sodium carbonate. In one embodiment the antacid is present at a level that is less than the amount recommended in the USP monograph in order to target temporary relief of reflux. This film may also include polydimethylsiloxanes. Examples of suitable polydimethylsiloxanes, which include, but are not limited to dimethicone and simethicone, are those disclosed in U.S. Pat. Nos. 4,906,478, 5,275,822, and 6,103,260, the contents of each is expressly incorporated herein by reference. As used herein, the term “simethicone” refers to the broader class of polydimethylsiloxanes, including but not limited to simethicone and dimethicone.

Ion exchange resins can be used for taste-masking the active ingredient. Preferred resins for this purpose are water-insoluble and consist of a pharmacologically inert organic or inorganic matrix containing covalently bound functional groups that are ionic or capable of being ionized under the appropriate conditions of pH. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix can also be, e.g., silica gel modified by the addition of ionic groups.

The covalently bound ionic groups may be strongly acidic (e.g., sulfonic acid), weakly acidic (e.g., carboxylic acid), strongly basic (e.g., quaternary ammonium), weakly basic (e.g., primary amine), or a combination of acidic and basic groups. In general, those types of ion exchangers suitable for use in ion exchange chromatography and for such applications as dcionization of water are suitable for use in these controlled release drug preparations. Such ion exchangers are described by H. F. Walton in “Principles of Ion Exchange” (pp. 312 343). The ion exchange resins useful in the present invention have exchange capacities below about 6 milliequivalents per gram (meq/g) and preferably below about 5.5 meq/g.

The resin is crosslinked with a crosslinking agent selected from difunctional compounds capable of crosslinking polystyrenes; these are commonly known in the art. Preferably, the crosslinking agent is a divinyl or polyvinyl compound. Most preferably the crosslinking agent is divinylbenzene. The resin is crosslinked to an extent of about 3 to about 20%, preferably about 4 to about 16%, more preferably about 6 to about 10%, and most preferably about 8% by weight based on the total resin. The resin is crosslinked with the crosslinking agent by means well known in the art.

The size of the ion exchange resins should preferably fall within the range of about 20 to about 200 micrometers. Particle sizes substantially below the lower limit are difficult to handle in all steps of the processing. Particle sizes substantially above the upper limit, e.g., commercially available ion exchange resins having a spherical shape and diameters up to about 1000 micrometers, are gritty in liquid dosage forms and have a greater tendency to fracture when subjected to drying-hydrating cycles.

Representative resins useful in this invention include AMBERLITE IRP-69 (obtained from Rohm and Haas) and Dow XYS-40010.00 (obtained from The Dow Chemical Company). Both are sulfonated polymers composed of polystyrene cross-linked with 8% of divinylbenzene, with an ion exchange capacity of about 4.5 to 5.5 meq/g of dry resin (H+-form). Their essential difference is in physical form. AMBERLITE IRP-69 comprises irregularly-shaped particles with a size range of 47 to 149 micrometers, produced by milling the parent, large-sized spheres of AMBERLITE IRP-120. The Dow XYS-40010.00 product comprises spherical particles with a size range of 45 to 150 micrometers. Another useful exchange resin, Dow XYS-40013.00, is a polymer composed of polystyrene cross-linked with 8% of divinylbenzene and functionalized with a quaternary ammonium group; its exchange capacity is normally within the range of approximately 3 to 4 meq/g of dry resin.

The most preferred resin is AMBERLITE IRP-69. However, in less preferred embodiments, the taste-masking agent need not be an ion exchange resin. In these embodiments, the taste-masking agent can be, e.g., magnesium trisilicate. See, e.g., U.S. Pat. Nos. 4,650,663 and 4,581,232 to Peters et al. Taste can also be masked by polymers, such as EUDRAGIT E (Rohm and Haas), and/or cellulosics, such as ethylcellulose, and the like.

The film-forming agent used in the films according to the present invention can be selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. A preferred film former is pullulan, in amounts ranging from about 0.01 to about 99 wt %, preferably about 30 to about 80 wt %, more preferably from about 45 to about 70 wt % of the film and even more preferably from about 60 to about 65 wt % of the film.

Unless specified otherwise, the term “wt %” as used herein with reference to the final product (i.e., the film, as opposed to the formulation used to create it), denotes the percentage of the total dry weight contributed by the subject ingredient. This theoretical value can differ from the experimental value, because in practice, the film typically retains some of the water and/or ethanol used in preparation.

In embodiments containing relatively high oil content, it is preferable to avoid substantial amounts of humectant in the film (and more preferable to have no humectant in the film), so as to avoid producing an overly moist, self-adhering film. In particular, it is preferred to formulate high oil content films with a plasticizing agent other than glycerin, which is also a humectant, and with a sweetener other than sorbitol, which is a mild humectant.

Saliva stimulating agents can also be added to the films according to the present invention. Useful saliva stimulating agents are those disclosed in U.S. Pat. No. 4,820,506. Saliva stimulating agents include food acids such as citric, lactic, malic, succinic, ascorbic, adipic, fumaric and tartaric acids. Preferred food acids are citric, malic and ascorbic acids. The amount of saliva stimulating agents in the film is from about 0.01 to about 12 wt %, preferably about 1 wt % to about 10 wt %, even more preferably about 2.5 wt % to about 6 wt %.

Plasticizers may be used in the film forming portion of the edible film. In the embodiment wherein the edible film comprises a detachable modified release matrix portion, a plasticizer may also be used. Preferred plasticizing agents include triacetin in amounts ranging from about 0 to about 20 wt %, preferably about 0 to about 10 wt %. Other suitable plasticizing agents include but are not limited to, polyethylene glycol; propylene glycol; glycerin; sorbitol; triethyl citrate; tributyl citrate; dibutyl sebecate; vegetable oils such as castor oil, rape oil, olive oil, and sesame oil; surfactants such as polysorbates, sodium lauryl sulfates, and dioctyl-sodium sulfosuccinatcs; mono acetate of glycerol; diacetate of glycerol; triacetate of glycerol; natural gums; triacetin; monoacetin, diacetin, acetyltributyl citrate; diethyloxalate; diethylmalate; diethyl fumarate; di ethylmalonate; dioctylphthalate; dibutylsuccinate; glyceroltributyrate; glycerol monostearate; hydrogenated castor oil; substituted triglycerides and glycerides; and mixtures thereof.

Preferred cooling agents include monomenthyl succinate, in amounts ranging from about 0.001 to about 2.0 wt %, preferably about 0.2 to about 0.4 wt %. A monomenthyl succinate containing cooling agent is available from Mane, Inc. Other suitable cooling agents include WS3, WS23, Ultracool II; or non-volatile coolers such as those sold under the tradename “Cooler No. 2” available from International Flavors and Fragrances (IFF) Corporation, and the like.

In one embodiment, a warming agent or sensate may be added. Warming agents are especially useful in improving the consumer experience in the delivery of an upper respiratory active ingredient such as pseudoephedrine, phenylephrine, dextromethorphan, diphenhydramine, chlorpheniramine, or menthol. Suitable warming agents may include but are not limited to capsaicin.

Preferred surfactants include mono and diglycerides of fatty acids and polyoxyethylene sorbitol esters, such as, Atmos 300 and Polysorbate 80. The surfactant can be added in amounts ranging from about 0.5 to about 15 wt %, preferably about 1 to about 5 wt % of the film. Other suitable surfactants include pluronic acid, sodium lauryl sulfate, and the like.

Preferred stabilizing agents include xanthan gum, locust bean gum and carrageenan, in amounts ranging from about 0 to about 10 wt %, preferably about 0.1 to about 2 wt % of the film. Other suitable stabilizing agents include guar gum and the like.

Preferred emulsifying agents include triethanolamine stearate, quaternary ammonium compounds, acacia, gelatin, lecithin, bentonite, veegum, and the like, in amounts ranging from about 0 to about 5 wt %, preferably about 0.01 to about 0.7 Wt % of the film.

Preferred thickening agents include methylcellulose, carboxyl methylcellulose, and the like, in amounts ranging from about 0 to about 20 wt %, preferably about 0.01 to about 5 wt %.

Preferred binding agents include starch, in amounts ranging from about 0 to about 10 wt %, preferably about 0.01 to about 2 wt % of the film.

Suitable sweeteners that can be included are those well known in the art, including both natural and artificial sweeteners. Suitable sweeteners include, e.g.: water-soluble sweetening agents such as monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin; water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like; dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexyen)-alanine, and the like; water-soluble sweeteners derived from naturally occurring water-soluble sweeteners, such as a chlorinated derivative of ordinary sugar (sucrose), known, for example, under the product description of sucralose; and protein based sweeteners such as thaumatoccous danielli (Thaumatin I and II).

In general, an effective amount of auxiliary sweetener is utilized to provide the level of sweetness desired for a particular composition, and this amount will vary with the sweetener selected. This amount will normally be 0.01% to about 10% by weight of the composition when using an easily extractable sweetener. The water-soluble sweeteners described in category A above, are usually used in amounts of about 0.01 to about 10 wt %, and preferably in amounts of about 2 to about 5 wt %. Other sweeteners are generally used in amounts of about 0.01 to about 10 wt %, with about 2 to about 8 wt % being preferred and about 3 to about 6 wt % being most preferred. These amounts may be used to achieve a desired level of sweetness independent from the flavor level achieved from any optional flavor oils used.

The flavorings that can be used include those known to the skilled artisan, such as natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in admixture. Commonly used flavors include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used.

Generally, any flavoring or food additive, such as those described in Chemicals Used in Food Processing, publication 1274 by the National Academy of Sciences, pages 63-258, may be used. Further examples of aldehyde flavorings include, but are not limited to acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof; and the like.

The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. In general, amounts of about 0.1 to about 30 wt % are useable with amounts of about 2 to about 25 wt % being preferred and amounts from about 8 to about 10 wt % are more preferred.

The compositions of this invention can also contain coloring agents or colorants. The coloring agents are used in amounts effective to produce the desired color. The coloring agents useful in the present invention, include pigments such as titanium dioxide, which may be incorporated in amounts of up to about 5 wt %, and preferably less than about 1 wt %. Colorants can also include natural food colors and dyes suitable for food, drug and cosmetic applications. These colorants are known as FD&C dyes and lakes. The materials acceptable for the foregoing spectrum of use are preferably water-soluble, and include FD&C Blue No. 2, which is the disodium salt of 5,5-indigotindisulfonic acid. Similarly, the dye known as Green No. 3 comprises a triphenylmethane dye and is the monosodium salt of 4-[4-N-ethyl-p-sulfobenzylamino)diphenyl-methylene]-[1-N-ethyl-N-p-sulfonium benzyl)-2,5-cyclo-hexadienimine] A full recitation of all FD&C and D&C dyes and their corresponding chemical structures may be found in the Kirk-Othmer Encyclopedia of Chemical Technology, Volume 5, Pages 857-884, which text is accordingly incorporated herein by reference.

The films can also include a triglyceride. Examples of triglycerides include vegetable oils such as corn oil, sunflower oil, peanut oil, olive oil, canola oil, soybean oil and mixtures thereof. A preferred triglyceride is olive oil. The triglyceride is added to the film in amounts from about 0.1 wt % to about 12 wt %, preferably in a range from about 0.5 wt % to about 9 wt %, of the film.

The films can include a preservative in amounts from about 0.001 wt % to about 5 wt %, preferably from about 0.01 wt % to about 1 wt % of the film. Preferred preservatives include sodium benzoate and potassium sorbate. Other suitable preservatives include, but are not limited to, salts of edetate (also known as salts of ethylenediaminetetraacetic acid, or EDTA, such as disodium EDTA) and parabens (e.g., methyl, ethyl, propyl or butyl-hydroxybenzoates, etc.) or sorbic acid. The preservatives listed above are exemplary, but each preservative must be evaluated on an empirical basis, in each formulation, to assure the compatibility and efficacy of the preservative. Methods for evaluating the efficacy of preservatives in pharmaceutical formulations are known to those skilled in the art.

The films can also include a polyethylene oxide compound. The molecular weight of the polyethylene oxide compound ranges from about 50,000 to about 6,000,000. A preferred polyethylene oxide compound is N-10 available from Union Carbide Corporation. The polyethylene oxide compound is added in amounts from about 0.1 wt % to about 5 wt %, preferably from about 0.2 wt % to about 4.0 wt % of the film.

The films can also include propylene glycol. The propylene glycol is added in amounts from about 1 wt % to about 20 wt %, preferably from about 5 wt % to about 15 wt % of the film.

Methods for preparing films according to the invention are capable of encapsulating the oil ingredients within the film-forming matrix and maintaining the integrity of the film, even when the film contains oils in amounts of 10 wt % or more.

In certain methods for preparing films according to the invention, the film-forming ingredients are mixed and hydrated with water separately from the water-soluble ingredients, which are mixed in aqueous solution separately from the organic ingredients and surfactants. In these methods, the final formulation is preferably produced by mixing the film-forming phase with the aqueous phase, then mixing in the organic phase, which includes surfactants, such as Polysorbate 80 and Atmos 300. This mass is mixed until emulsified. In other embodiments, the aqueous and film forming phases are combined into a single phase by dissolving the water-soluble ingredients in the water and then adding the gums to hydrate. The organic phase is then added to this single aqueous phase.

The resulting formulation is cast on a suitable substrate and dried to form a film. The film is preferably air-dried or dried under warm air and cut to a desired dimension, packaged and stored. The film can contain from about 0.1% to about 10 wt % moisture, preferably from about 3% to about 8 wt % moisture, even more preferably from about 4 to about 7 wt % moisture.

The film-forming phase can include pullulan and stabilizing agents such as xanthan gum, locust bean gum and carrageenan. These ingredients are mixed and then hydrated in water for about 30 to about 48 hours to form a gel. The water is preferably heated to a temperature of about 25 to about 45° C. to promote hydration. The amount of water is about 40 to 80% of the gel. The resulting hydrated gel is then chilled to a temperature of about 20 to about 30° C. for about 1 to about 48 hours. The water is preferably deionized.

In preferred embodiments, the aqueous phase includes water heated to a temperature of about 60 to 90° C., preferably 70 to 80° C., and ingredients such as the pharmaceutically active agent, ion exchange resin (or other masking agent), coloring agent, preservative and sweetener. The water is preferably deionized and the amount of water used is about 5 to about 80 wt % of the final gel mixture.

The pharmaceutically active agent can be incorporated into or onto the ion exchange resin for taste-masking purposes. Other taste-masking methods, such as coating, are known in the art.

Adsorption of the pharmaceutically active agent onto the ion exchange resin particles to form the pharmaceutically active agent/resin complex is a well-known technique as shown in U.S. Pat. Nos. 2,990,332 and 4,221,778. In general, the pharmaceutically active agent is mixed with an aqueous suspension of the resin, and in less preferred embodiments, the complex is then washed and dried. Adsorption of pharmaceutically active agent onto the resin may be detected by measuring a change in the pH of the reaction medium, or by measuring a change in concentration of sodium or pharmaceutically active agent.

Binding of pharmaceutically active agent to resin can be accomplished according to four general reactions. In the case of a basic pharmaceutically active agent, these are: (a) resin (Na-form) plus pharmaceutically active agent (salt form); (b) resin (Na-form) plus pharmaceutically active agent (as free base); (c) resin (H-form) plus pharmaceutically active agent (salt form); and (d) resin (H-form) plus pharmaceutically active agent (as free base). All of these reactions except (d) have cationic byproducts, by competing with the cationic pharmaceutically active agent for binding sites on the resin, reduce the amount of pharmaceutically active agent bound at equilibrium. For basic pharmaceutically active agents, stoichiometric binding of pharmaceutically active agent to resin is accomplished only through reaction (d).

Four analogous binding reactions can be carried out for binding an acidic pharmaceutically active agent to an anion exchange resin. These are: (a) resin (Cl-form) plus pharmaceutically active agent (salt form); (b) resin (Cl-form) plus pharmaceutically active agent (as free acid); (c) resin (OH-form) plus pharmaceutically active agent (salt form); and (d) resin (OH-form) plus pharmaceutically active agent (as free acid). All of these reactions except (d) have ionic by-products and the anions generated when the reactions occur compete with the anionic pharmaceutically active agent for binding sites on the resin with the result that reduced levels of pharmaceutically active agent are bound at equilibrium. For acidic pharmaceutically active agents, stoichiometric binding of pharmaceutically active agent to resin is accomplished only through reaction (d). The binding may be performed, for example, as a batch or column process, as is known in the art.

In less preferred embodiments, the adsorption complex, including pharmaceutically active agent and resin, is collected and washed with ethanol and/or water to insure removal of any unadsorbed pharmaceutically active agent. The complexes are usually air-dried in trays at room or elevated temperature.

The ratio of the pharmaceutically active agent adsorbate to ion exchange resin adsorbent in the adsorption complex is about 1:3 to about 3:1, preferably about 1:2 to about 2:1, most preferably about 1:1. The only limit to using ratios in excess of 1:3 is an economic and aesthetic one.

The amount of the pharmaceutically active agent adsorbed to the ion exchange resin is in the range from about 25 to about 75% by weight of the pharmaceutically active agent/resin adsorption complex (hereinafter referred to as the “pharmaceutically active agent/resin complex” or “complex”). More preferably, the amount of the pharmaceutically active agent adsorbed to the ion exchange resin is in the range from about 33 to about 77% by weight of the pharmaceutically active agent/resin complex. Most preferably, the amount of the pharmaceutically active agent adsorbed to the ion exchange resin is in the range from about 40 to about 60% by weight of the pharmaceutically active agent/resin complex.

The amount of pharmaceutically active agent/resin complex in the formulation is adjusted to deliver a predetermined dose of the pharmaceutically active agent over a predetermined period of time.

For example, a preferred antitussive film of the invention is administered at one dose every 12 hours to deliver a pharmaceutically effective amount of dextromethorphan over a period of approximately 12 hours to a patient in need of such administration. A typical adult dose of a film of the invention measuring 1″×1.25″ (2.54 cm×3.18 cm) weighs about 60 to about 190 mg and contains about 20 to about 130 mg of pharmaceutically active agent/resin complex to deliver about 5 to about 65 mg of pharmaceutically active agent (e.g., dextromethorphan hydrobromide) when the average pharmaceutically active agent: ion exchange resin ratio is about 1:1.

In embodiments, a certain percentage of the films disclosed herein can contain non-coated pharmaceutically active agent/resin complexes. The remaining pharmaceutically active agent/resin complexes are further characterized by the presence of a coating. In the preferred embodiment of the present invention, about 20 to about 80% of the pharmaceutically active agent/resin complexes in the sustained-release compositions are coated, most preferably about 40 to about 60% of the pharmaceutically active agent/resin complexes. The coating is a water-permeable, diffusion barrier coating material. The presence of a coating allows one to selectively modify the dissolution profile as desired of a pharmaceutical composition comprising the pharmaceutically active agent/resin complexes of the present invention.

The coating materials can in general be any of a large number of conventional natural or synthetic film-forming materials used singly, in admixture with each other, and in admixture with plasticizers, pigments, etc. with diffusion barrier properties and with no inherent pharmacological or toxic properties. In general, the major components of the coating should be insoluble in water, and permeable to water and pharmaceutically active agent. However, it might be desirable to incorporate a water-soluble substance, such as methyl cellulose, to alter the permeability of the coating, or to incorporate an acid-insoluble, base-soluble substance to act as an enteric coating. The coating materials may be applied as a suspension in an aqueous fluid or as a solution in organic solvents. Suitable examples of such coating materials are described by R. C. Rowe in Materials used in Pharmaceutical Formulation. (A. T. Florence, editor), Blackwell Scientific Publications, Oxford, 1 36(1984), incorporated by reference herein. Preferably the water-permeable diffusion barrier is selected from the group consisting of ethyl cellulose, methyl cellulose and mixtures thereof.

Most preferably, the coating material is SURELEASE, manufactured by Colorcon which is water based ethyl cellulose latex, plasticized with dibutyl sebacate or with vegetable oils. Other non-limiting coating materials included within the scope of the present invention are AQUACOAT, manufactured by FMC Corporation of Philadelphia, which is ethylcellulose pseudolatex; solvent based ethylcellulose; shellac; zein; rosin esters; cellulose acetate; EUDRAGIT, manufactured by Rohm and Haas of Philadelphia, which are acrylic resins; silicone elastomers; poly(vinyl chloride) methyl cellulose; and hydroxypropylmethyl cellulose.

Conventional coating solvents and coating procedures (such as fluid bed coating and spray coating) can be employed to coat the particles. Techniques of fluid bed coating are taught, for example, in U.S. Pat. Nos. 3,089,824, 3,117,027, and 3,253,944. The coating is normally applied to the pharmaceutically active agent/resin complex, but alternatively can be applied to the resin before complexing with the pharmaceutically active agent. Non-limiting examples of coating solvents include ethanol, a methylene chloride/acetone mixture, coating emulsions, methyl acetone, tetrahydrofuran, carbonetetrachloride, methyl ethyl ketone, ethylene dichloride, trichloroethylene, hexane, methyl alcohol, isopropyl alcohol, methyl isobutyl ketone, toluene, 2-nitropropane, xylene, isobutyl alcohol, n-butyl acetate.

It is preferred that the coated pharmaceutically active agent/resin complexes are coated in the range from about 40 to about 70% w/w pharmaceutically active agent/resin complex. More preferably, the pharmaceutically active agent/resin complex is coated in the range from about 45 to about 55% w/w pharmaceutically active agent/resin complex. Most preferably, the pharmaceutically active agent/resin complex is coated about 50% w/w pharmaceutically active agent/resin complex. Variation in the amount of coating and/or the use of coated/uncoated complex mixtures can be employed to selectively modify the dissolution profile as desired.

The average particle sizes of the non-hydrated coated and uncoated pharmaceutically active agent/resin complexes is about 60 to about 200 and about 60 to about 250 micrometers, respectively. More preferably, average particle sizes of the coated pharmaceutically active agent/resin complexes is between about 70 and about 190 micrometers, and most preferably about 70 to about 180 micrometers. More preferably, average particle sizes of the uncoated pharmaceutically active agent/resin complexes is between about 55 and about 160 micrometers, and most preferably about 60 to about 150 micrometers. It is desirable that about 85%, preferably about 95%, and most preferably about 98% of the resin particles have sizes within the ranges set forth above. Adjustments within these ranges can be made to accommodate desired aesthetic qualities of the final formulation product. It is more preferable that the resin dextromethorphan complex have particle sizes within these ranges as well.

In certain embodiments the edible film may incorporate microgel beads, which are liquid filled in semi-solid filled. The edible film may comprise a first active ingredient where the liquid filled beads comprise a second active ingredient. The edible film form of this invention has the added advantage of not using a compression step, as do tablets forms, allowing for the use of liquid or semisolid filled particles or beads that are deformable, since they will not rupture upon compression. These beads may be coated with gelling substances such as but not limited to gelatin, gellan gum, xanthan gum, agar, locust bean gum, carrageenan; polymers or polysaccharides such as but not limited to sodium alginate, calcium alginate, hypromellose, hydroxypropyl cellulose and pullulan; and starches; with or without the addition of plasticizers such as but not limited to glycerin, polyethylene glycol, propylene glycol, triacetin, triethyl citrate and tributyl citrate. The active ingredient may be dissolved, suspended or dispersed in a filler material such as but not limited to high fructose corn syrup, sugars, glycerin, polyethylene glycol, propylene glycol, or oils such as but not limited to vegetable oil, olive oil, or mineral oil. In one embodiment the bead does not contain an active ingredient, but contains flavorants to facilitate swallowing of the entire dosage form. In this embodiment the edible film may contain other suspended or dissolved actives. The average mean diameter of these microgel beads may be from about 100 microns to about 3000 microns.

In certain embodiments the particle coating polymer systems may be used in order to taste-mask the active ingredients. Suitable particle coatings may be made up of water insoluble polymers such as cellulose acetate combined with a pore forming polymer material such as polyvinyl pyrrolidone, hydroxypropyl cellulose, polymethacrylic polymers and co-polymers or hypromellose. Suitable polymethacrylic co-polymers for use as pore formers include those such as cationic polymers with dimethylaminoethyl methacrylate as a functional group, which are also sold under the tradename Eudragit E100. In this embodiment the preferred coating level, by weight of the coated particle, is from about 2 percent to about 30 percent, e.g. 5 percent to about 30 percent. In one embodiment, a suitable plasticizer may be used in an amount, based upon the total dry weight of the coating, from about 0.1% to about 40%, e.g. about 1% to about 30% or from about 5% to about 20%. In this embodiment the weight ratio of water insoluble polymer to pore former is about 50:50 to about 95:5, or about 70:30 to about 95:5.

In certain embodiments, it is possible to hydrate the film-forming ingredients and combine all of the ingredients without heating. This method comprises dissolving the water-soluble ingredients in water to form an aqueous mixture; mixing the film-forming ingredients in powder form to form a powder mixture; adding the powder mixture to the aqueous mixture to form a hydrated polymer gel; stirring the hydrated polymer at room temperature for about 30 minutes to about 48 hours; mixing the cooling agent, menthol and any other oils to form an oil mixture; adding the oil mixture to the hydrated polymer gel and mixing until uniform; deaerating the film until air bubbles are removed, casting the uniform mixture on a suitable substrate; and drying the cast mixture to form a film. This method hydrates the film-forming ingredients without heating the water, which can reduce energy costs in the manufacturing process and undesirable losses of volatile ingredients to evaporation. Further, mixing the oils in two steps minimizes the amount of flavor lost.

In one embodiment, as the strip material is deposited and lined onto the backing material, prior to the drying process, the active ingredient is portioned using a powder feeder device or powder jet. The backing material may be made of paper, plastic or metal.

In one embodiment, a single layer film can be manufactured by a coating process utilizing a backing A casting station transfers bulk solution or suspension from the mixing vessel into a thin film on the surface of a release liner. The release liner can be made of a variety of materials including but not limited to paper, polypropylene, plastics, polymer films, steel, or aluminum. This is followed by a drying or curing process to remove carrier solvents usually using a multi-zone dryer for efficiency. Suitable solvents may include aqueous systems such as purified water or pH buffering systems; or alternatively, organic solvents such as ethanol, methanol, acetone or mixtures thereof, including mixtures of water and organic solvent. In this embodiment, the line speed which feeds the roll of the film, the air temperature, and velocity are controlled to optimize drying. In addition, in this embodiment, the film with liner is rolled and slit, and the final product rendered to its optimum dimensions and packaged.

In one embodiment, a bilayer film can be manufactured by a coating process utilizing a backing A casting station transfers bulk solution or suspension from the mixing vessel into a thin film on the surface of a release liner. This is followed by a drying or curing process to remove carrier solvents usually using a multi-zone dryer for efficiency. In this embodiment, suitable liner and solvent materials are similar to those described above. The line speed, air temperature, and velocity are controlled to optimize drying. In this embodiment, the film with liner can be coated with the second layer of the bilayer film from a casting station that could transfer the bulk solution or suspension from the second mixing vessel onto the surface of the former film. This could be followed by rolling, slitting, and packaging as described above.

In another embodiment, the strip is formed using extrusion or molding and is substantially free of the use of solvents. In this embodiment, solvents include water or organic solvents such as alcohol, ethanol, methanol, isopropanol acetone of methylene chloride and substantially free can be defined as less than 10 percent, e.g. less than 5 percent, e.g. less than 1 percent of solvent by weight of the total weight of strip material. If the strip is produced by solvent free molding or extrusion, the active ingredient can be advantageously placed in certain places along the strip by co-extruding the active or a portion of strip material containing active only on the sides of the strip. This can be achieved using a separate supply and feed line containing active ingredient and co-extruded at the point of where the main strip extrusion material, which contains no active or a second active ingredient, is delivered.

The present invention is further described by the following non-limiting examples. The scope of the invention is defined by reference to the following claims.

### Example 1

### Preparation of Thin Film

The ingredients listed in Table 1 are combined to provide an example of an antitussive film in accordance with the following procedure:

Water is heated to 75° C. Uncoated dextromethorphan hydrobromide is dissolved with mixing in water, while maintaining the mixture at a temperature of 75° C. AMBERLITE resin is then mixed into the water with heating for 4 to 5 hours at 70-80° C. Heating is stopped, water lost to heating is added, and potassium sorbate and sweetener are dissolved in the water with mixing;

The film forming ingredients including the xanthan gum, locust bean gum, carrageenan and pullulan are mixed in a separate container with rapid mixing (at approximately 100 RPM) using a lab scale Lightning mixer for 15 minutes, followed by mixing for at least 12 hours at approximately 25 RPM to produce a gum/thickener mixture;

Menthol is mixed in alcohol (USP) carrier in a separate container. Physcool is dissolved therein with mixing. MAG, PolySorbate 80, Atmos 300 and flavors are added to the alcohol mixture and then added to the gum/thickener mixture above and mixed at 25 RPM. Glycerin and mannitol are added to this mixture at 25 RPM, mixing continues;

The resulting preparation is poured into a rectangular mold and allowed to cast a film. Phenylephrine HCl is then sprinkled evenly onto a top portion of the film equal to ¼ of the surface area of the mold. The active-coated film is then segmented into 1.5″×0.75″ portions at a weight of 78+/−5 mg, resulting in a thin film dosage form with dextromethorphan evenly distributed throughout the film and phenylephrine hydrochloride only on one portion of the form.

### Example 2

### Preparation of Arrow and Tapered Films

Similar films are cast using the solution from Example 1 in two other types of molds to indicate direction for ingestion:

Phenylephrine hydrochloride is sprinkled onto the arrow portion only of an arrow shaped film with the tail end which is 0.75 inches long and the arrow is 0.50 inches long and 0.5 inches wide;

Phenylephrine hydrochloride is sprinkled onto the top end of a tapered film that is 1.5 inches long and 0.75 inches wide at one end and 0.25 inches long at the top end;

A film of the same shape that is used in Example 1 is prepared wherein 60 percent of the phenylephrine (4.5 mg) is sprinkled on the top third of the strip, 30 percent of the phenylephrine (2.25 mg) is sprinkled on the middle third of the strip and 10 percent of the phenylephrine (0.75 mg) is sprinkled onto the bottom third of the strip. This is designed by creating a gradient effect with the uncoated, more bitter active, so that the portion of the strip with the heaviest drug loading is ingested first and felt on the back of the tongue only.

TABLE 1
Upper Respiratory Edible Film (Strip) Formulation

| | | | % w/w in | |
|---|---|---|---|---|
| Material | G/batch | % w/w | active film | Mg/dose |
| Coated | 4.770 | 4.770 | 19.230 | 15.00 |
| Dextromethorphan (32%) | | | | |
| Amberlite IRP69 | 5.086 | 5.086 | 20.510 | 16.00 |
| Xanthan Gum | 0.030 | 0.030 | 0.121 | 0.094 |
| Locust Bean Gum | 0.035 | 0.035 | 0.141 | 0.110 |
| Carrageenan | 0.150 | 0.150 | 0.605 | 0.472 |
| Pullulan | 8.630 | 8.630 | 34.800 | 27.144 |
| Potassium Sorbate | 0.030 | 0.030 | 0.121 | 0.094 |
| Sucralose | 0.477 | 0.477 | 1.923 | 1.500 |
| Purified Water | 70.20 | 70.20 | | NA |
| Alcohol USP | 5.00 | 5.00 | | NA |
| Physcool | 0.050 | 0.050 | 0.201 | 0.157 |
| Menthol | 0.750 | 0.750 | 3.026 | 2.360 |
| Raspberry Flavor | 0.250 | 0.250 | 1.010 | 0.786 |
| Peppermint Flavor | 0.050 | 0.050 | 0.201 | 0.157 |
| Mono ammonium | 0.005 | 0.005 | 0.021 | 0.016 |
| glycyrrhizinate (MAG) | | | | |
| Polysorbate 80 | 0.175 | 0.175 | 0.705 | 0.550 |
| Atmos 300 | 0.175 | 0.175 | 0.705 | 0.550 |
| Glycerin | 0.750 | 0.750 | 3.026 | 2.360 |
| Mannitol USP | 1.001 | 1.001 | 4.038 | 3.15 |
| Phenylephrine HCl | 2.385 | 2.385 | 9.615 | 7.50 |
| | 100.0 | 100.0 | 100.0 | 78.00 |

### Example 3

### Preparation of Film Containing Topical Anesthetic & Menthol

The ingredients listed in Table 2 are combined to provide an example of a sore throat treating film in accordance with the following procedure:

Water is heated to 75° C. Potassium sorbate and sweetener are dissolved in the water with mixing;

The film forming ingredients including the xanthan gum, locust bean gum, carrageenan and pullulan are mixed in a separate container with rapid mixing (at approximately 100 RPM) using a lab scale Lightning Mixer for 15 minutes, followed by mixing for at least 12 hours at approximately 25 RPM to produce a gum/thickener mixture;

Menthol is mixed with alcohol (USP) carrier in a separate container. Physcool is dissolved therein with mixing. MAG, PolySorbate 80, Atmos 300 and flavors are added to the mixture and then added to the gum/thickener mixture and mixed at 25 RPM. Glycerin and mannitol are added to this mixture at 25 RPM and continued to mix;

The resulting preparation is poured onto a rectangular mold a mold and cast as a shaped film. Benzocaine is then sprinkled evenly onto a top portion of the shaped film that is equal to ¼ of the surface area of the mold and allowed to dry in an oven set at 30° C. for approximately 12 hours. The active-coated film is then segmented into 1.5″×0.75″ portions at a weight of 78+/−5 mg, resulting in a thin film dosage form with benzocaine distributed on only one top portion of the film.

TABLE 2
Benzocaine Edible Film (Strip) Formulation

| | | | % w/w in | |
|---|---|---|---|---|
| Material | G/batch | % w/w | active film | Mg/dose |
| Benzocaine | 2.480 | 2.480 | 10.00 | 6.00 |
| Xanthan Gum | 0.039 | 0.039 | 0.157 | 0.094 |
| Locust Bean Gum | 0.045 | 0.045 | 0.183 | 0.110 |
| Carrageenan | 0.195 | 0.195 | 0.787 | 0.472 |
| Pullulan | 14.053 | 14.053 | 56.667 | 34.004 |
| Potassium Sorbate | 0.039 | 0.039 | 0.157 | 0.094 |
| Sucralose | 0.620 | 0.620 | 2.50 | 1.500 |
| Purified Water | 70.200 | 70.200 | NA | NA |
| Alcohol USP | 5.000 | 5.000 | NA | NA |
| Physcool | 0.065 | 0.065 | 0.262 | 0.157 |
| Menthol | 4.133 | 4.133 | 16.667 | 10.00 |
| Raspberry Flavor | 0.325 | 0.325 | 1.310 | 0.786 |
| Peppermint Flavor | 0.065 | 0.065 | 0.262 | 0.157 |
| Mono ammonium | 0.007 | 0.007 | 0.027 | 0.016 |
| glycyrrhizinate | | | | |
| PolySorbate 80 | 0.227 | 0.227 | 0.917 | 0.550 |
| Atmos 300 | 0.227 | 0.227 | 0.917 | 0.550 |
| Glycerin | 0.975 | 0.975 | 3.930 | 2.360 |
| Mannitol USP | 1.302 | 1.302 | 5.250 | 3.15 |
| | 100.0 | 100.0 | 100.0 | 60.00 |

### Example 4

### Preparation of Immediate Release and Modified Release Edible Film

A edible film dispersion is prepared containing hydroxypropyl methylcellulose (HPMC) having a viscosity of about 4000 mPa s in 2% aqueous solution [commercially available from Dow Chemical as METHOCEL K4M]; Kappa Carrageenan, and remaining materials described in Table 3 in purified water. The solution has a solids concentration of 18.0%.

First, carrageenan, phenylephrine, sucralose, Physcool, Peppermint flavor and glycerin are dispersed in room temperature water with an electric mixer equipped with a propeller style blade to form a liquid carrier. Next, the carrageenan/water dispersion is heated to about 80° C. with continued mixing. Next, the HPMC and pullulan are dispersed in the liquid carrier with the propeller mixer, and mixing continued to maintain the HPMC in a suspended state at 80° C.

Next, approximately 314.52 mg of the Immediate Release Upper Respiratory edible film dispersion formulation (equivalent to 78 mg of solids) in Table 1 is poured into a mold held at room temperature. About 333.33 mg of the modified release edible film formulation (equivalent to 60 mg of solids) from Table 3 is poured on top of the immediate release film, such that approximately 2 mm of the two film portions overlap. The composition is allowed to dry at approximately 30° C. for 12 hours and removed from the mold as a finished dosage form.

TABLE 3
Modified Release Edible Film (Strip) Formulation

| | | | % w/w in | |
|---|---|---|---|---|
| Material | G/batch | % w/w | active film | Mg/dose |
| Phenylephrine | 4.500 | 4.500 | 25.00 | 15.00 |
| Kappa Carrageenan | 0.195 | 0.195 | 1.380 | 0.826 |
| HPMC K4M | 6.000 | 6.000 | 33.333 | 20.00 |
| Pullulan | 6.000 | 6.000 | 33.333 | 20.00 |
| Sucralose | 0.450 | 0.450 | 2.50 | 1.500 |
| Purified Water | 82.00 | 82.00 | NA | NA |
| Physcool | 0.047 | 0.047 | 0.262 | 0.157 |
| Peppermint Flavor | 0.047 | 0.047 | 0.262 | 0.157 |
| Glycerin | 0.707 | 0.707 | 3.930 | 2.360 |
| | 100.0 | 100.0 | 100.0 | 60.00 |

1. An edible film-strip comprising two or more segmented portions, a therapeutic active ingredient that is distributed on at least one of the segmented portions.

2. An edible film strip of claim 1 wherein the active is present in less than 50% of the total cross sectional surface area of a major face of said film.

3. An edible film strip of claim 1 wherein a segmented portion comprises, within a part of said segmented portion of a length of at least 2 millimeters to a maximum of at least 6 millimeters, a concentration of active ingredient which is 10 percent greater by weight of total active than in a separate part equal in length from a separate portion of the film.

4. An edible film-strip according to claim 1 wherein one or more segmented portions of the film is substantially free of active ingredient.

5. An edible film-strip comprising first and second portions and first and second active ingredients wherein the first active ingredient has a higher bitterness level than the second active ingredient and is contained only on the first portion of the edible film-strip and the second active ingredient is contained only on the second portion of the edible film-strip.

6. The edible film-strip of claim 1 wherein the film is tapered such that the second portion has a width on its major face that is less than 90% of the width of the first portion of its major face.

7.-14. (canceled)

15. An edible film-strip comprising a first portion and a second portion and at least different two pharmaceutically active ingredients, wherein the first pharmaceutically active ingredient is contained on the first portion that provides an immediate release portion of the first pharmaceutically active ingredient in a dissolution medium and the second active ingredient is contained on the second portion that provides a modified release of the second pharmaceutically active ingredient in a dissolution medium, wherein the second portion detaches from the first portion upon ingestion.

16. The edible film-strip of claim 15 wherein the film is tapered such that the second portion has a width on its major face that is less than 90% of the width of the first portion of its major face.

17. An edible bilayer film-strip, wherein the second layer comprises a therapeutic active ingredient.

18. An edible bilayer film-strip of claim 17, wherein the first layer is substantially free of a therapeutic active ingredient.

19. An edible bilayer film of claim 17 wherein the first layer and the second layer comprise the same active ingredient, but the second layer comprises a different amount of active ingredient than the amount in the first layer.

20. An edible film comprising a first therapeutic active ingredient and microgel liquid filled beads.

21. An edible film of claim 18 wherein the liquid filled beads are substantially free of therapeutic active ingredient.

22. An edible film of claim 18 wherein the film comprises a first active ingredient and wherein the microgel beads comprise a second active ingredient.

23. A method of reducing the bitterness of at least one active ingredient using the edible film of claim 1.
